(19) United States
(12) Patent Application Publication
(10) Pub. No.: US 2012/0253445 A1
(43) Pub. Date: Oct. 4, 2012

(54) MRI compatible conductor system for catheter and stimulation leads

(75) Inventor: Thomas P. Osypka, Palm Harbor, FL (US)

(73) Assignee: Oscor Inc., Palm Harbor, FL (US)

(21) Appl. No.: 13/308,010

(22) Filed: Nov. 30, 2011

Related U.S. Application Data
(60) Provisional application No. 61/470,138, filed on Mar. 31, 2011.

(51) Int. Cl. A61N 1/04; H05K 13/00

(52) U.S. Cl. 607/148; 607/115; 29/592.1

(57) ABSTRACT

A medical device compatible with magnetic resonance imaging including an elongated tubular body defining a longitudinal axis and having opposed proximal and distal end portions. The elongated body including a plurality of coaxial layers including a radially inner-most insulative layer, at least one inner conductive layer, and a radially outer-most insulative layer. The at least one conductive layer is defined by a non-ferromagnetic conductive material that is compatible with magnetic resonance imaging.

### CROSS Reference To RELATED APPLICATIONS

This application claims priority to U.S. Patent Application Ser. No. 61/470,138 filed Mar. 31, 2011, which is incorporated herein by reference in its entirety.

### FIELD OF THE INVENTION

The subject invention is directed generally to limiting MRI induced current in a stimulation lead such as a neurostimulation lead, a cardiac stimulation lead, and the like.

### BACKGROUND OF THE RELATED ART

Magnetic resonance imaging (MRI) is a non-invasive method used to render images of the inside of an object. It is primarily used in medical imaging to demonstrate pathological or other physiological alterations of living tissues. An illustrative MRI device is shown in FIG. 1. Cardiac pacing and stimulation leads generally include one or more electrical conductors typically fabricated from stainless steel or MP35N, and typically either in the form of a conductor coil or multi-stranded wire. These conductors electrically connect the proximal lead connector electrode with the distal lead electrode. This design allows for a reliable lead design. However, a significant disadvantage of this design is it is not MRI compatible due to heat absorption principals from the MRI scan.

Pacemakers and pacemaker lead systems are generally considered incompatible with MRI procedures. It is noted highly specialized protocols have been developed to permit scanning of select pacing devices. This notwithstanding, several cases of arrhythmias or death have been reported in patients with pacemakers who have undergone MRI scanning without appropriate precautions. Other electronic implants also have varying incompatibility issues, depending upon scanner technology, implant properties, scanning protocols and anatomy being imaged.

Though pacemakers and lead systems receive significant attention, it should also be noted that many other forms of medical or neuro-stimulation implants may be contraindicated for MRI scans. These may include Vagus nerve stimulators, implanted cardio-defibrillators (ICD), loop recorders, insulin pumps, cochlear implants, deep brain stimulators and many others medical catheters.

Thus, there is a need for the design and manufacture of a MRI compatible conductor system to be used in implantable stimulation leads and catheters to make them MRI compatible.

### SUMMARY OF THE INVENTION

The subject invention is directed to a new and useful medical component compatible for use with medical Magnetic Resonance imaging (MRI) procedures.

In one aspect of the invention, the standard conductor coils or filars of a lead and/or catheter device are replaced by an electrical active coating sputtered (e.g., a vapor deposition process) preferably on a silicone or biocompatible polymer tubing or a surface applied electroploymerized conductive polymer. It is to be appreciated a primer or carrying layer may be applied on the silicone or polymer tubing for the sputtered or applied conductive material to adhere in certain applications. Thus, a lead conductor as found in prior art lead and/or catheter medical devices is replaced with a silicone or polymer tubing, on which a conductive layer is positioned via an aforesaid sputtering or adhesion process. This conductive layer is then preferably electrically coupled to an electrode affixed to each of the distal and proximal end portions of the lead and/or catheter medical device.

It is to be appreciated the sputtered or applied conductive layer is preferably a non-ferromagnetic conductive material that is compatible with magnetic resonance imaging such as Titanium Nitrite, or Iridium Oxide or other sputtered coatings providing electrical conductivity while not having any undesired effects through exposure to a MRI process. It is further to be understood and appreciated the distal and proximal electrodes are fabricated from an MRI compatible non-ferromagnetic electrical conductive material (such as Titanium or Carbon). Additionally, the electrical resistance of the conductive layer coating extending between each proximal and distal electrode has a resistance less than 100 Ohms.

In one illustrated embodiment of the invention, what is provided is a medical device compatible with MRI procedures which includes an elongated tubular body defining a longitudinal axis and having opposed proximal and distal end portions. The elongated body includes a plurality of coaxial layers including a radially inner-most insulative layer, at least one inner conductive layer, and a radially outer-most insulative layer. It is to be understood and appreciated the at least one conductive layer is defined by a non-ferromagnetic conductive material that is compatible with MRI procedures.

In another illustrated embodiment of the invention, what is provided is a medical device compatible with magnetic resonance imaging including an elongated tubular body defining a longitudinal axis and having opposed proximal and distal end portions. The elongated body includes a radially inner insulative layer, an inner medial conductive layer surrounding the inner insulative layer, a medial insulative layer surrounding the inner medial conductive layer, an outer medial conductive layer surrounding the medial insulative layer and a radially outer insulative layer surrounding the outer medial conductive layer. It is to be understood and appreciated the inner and outer medial conductive layers are defined by a non-ferromagnetic conductive material that is compatible with magnetic resonance imaging which is applied to at least a portion of the elongated tubular body by a sputtering process. Further included is at least one distal electrode associated with the distal end portion of the tubular body and a proximal electrode associated with the proximal end portion of the tubular body, wherein at least one conductive layer is connected to the distal and proximal electrodes.

### BRIEF DESCRIPTION OF THE DRAWINGS

The objects and features of the invention can be understood with reference to the following detailed description of an illustrative embodiment of the present invention taken together in conjunction with the accompanying drawings in which:

FIG. 1 illustrates a MRI device depicting an environment of use for the present invention;

FIG. 2 illustrates the present invention conductor coupling to a pacemaker device;

FIG. 3 illustrates a perspective view of the present invention conductor;

FIG. 4 illustrates a cross-sectional view taken along line 4-4 of FIG. 3;

FIG. 5 illustrates a cross-sectional view taken along line 5-5 of FIG. 4;

FIG. 6 illustrates a cross-sectional view of another illustrated embodiment of the present invention;

FIG. 7 illustrates a cross-sectional view taken along line 7-7 of FIG. 6;

FIG. 8 illustrates a cross-sectional view of yet another illustrated embodiment of the present invention; and

FIG. 9 illustrates a cross-sectional view taken along line 9-9 of FIG. 8.

### DETAILED DESCRIPTION OF THE PREFERRED EMBODIMENTS

The present invention is now described more fully with reference to the accompanying drawings, in which an illustrated embodiment of the present invention is shown. The present invention is not limited in any way to the illustrated embodiment as the illustrated embodiment described below is merely exemplary of the invention, which can be embodied in various forms, as appreciated by one skilled in the art. Therefore, it is to be understood that any structural and functional details disclosed herein are not to be interpreted as limiting, but merely as a basis for the claims and as a representative for teaching one skilled in the art to variously employ the present invention. Furthermore, like reference numerals identify similar structural elements or features of the subject invention, and the terms and phrases used herein are not intended to be limiting but rather to provide an understandable description of the invention.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs.

Although any methods and materials similar or equivalent to those described herein can also be used in the practice or testing of the present invention, exemplary methods and materials are now described. All publications mentioned herein are incorporated herein by reference to disclose and describe the methods and/or materials in connection with which the publications are cited.

It must be noted that as used herein and in the appended claims, the singular forms “a”, “an,” and “the” include plural referents unless the context clearly dictates otherwise. Thus, for example, reference to “a stimulus” includes a plurality of such stimuli and reference to “the signal” includes reference to one or more signals and equivalents thereof known to those skilled in the art, and so forth.

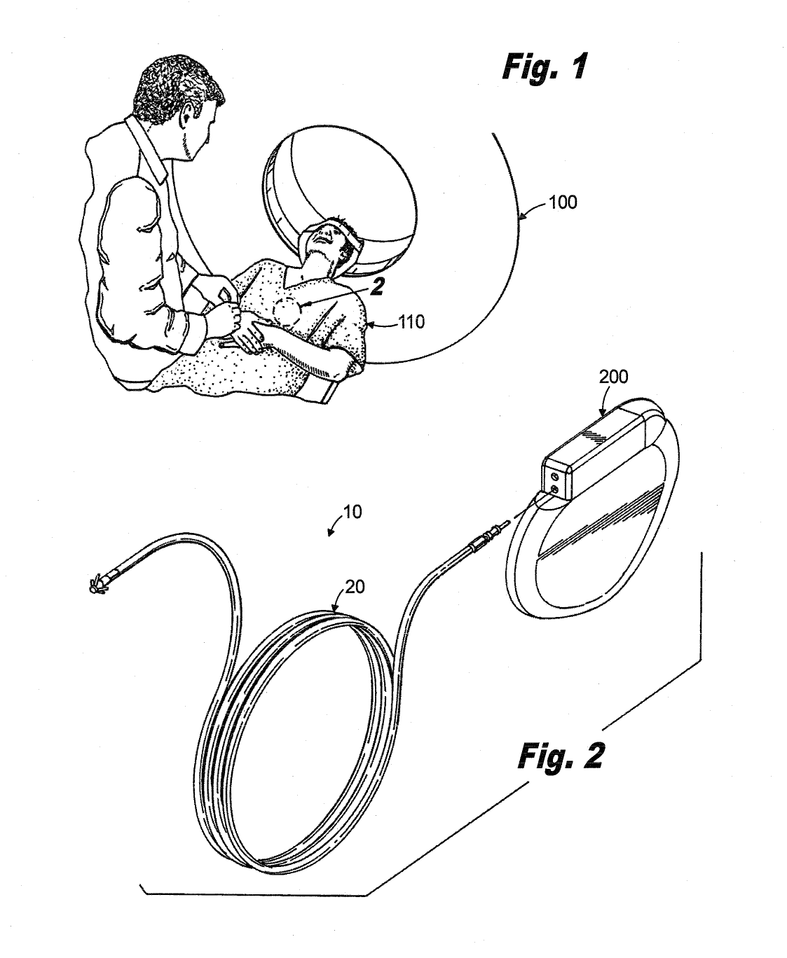

With reference now to the drawings, there is illustrated in FIG. 1 an MRI device 100 depicting an environment of use for the present invention conductor. FIG. 1 depicts a patient 110 undergoing an MRI procedure, which for illustrative purposes, the patient preferably has a pacemaker device 200 coupled to the present invention implantable lead instrument 10. FIG. 2 illustrates an exemplary pacemaker device 200 adapted and configured to couple to the present invention implantable lead instrument 10. It is to be appreciated and understood that since the aforementioned MRI device 100 and pacemaker device 200 do not constitute the invention but constitute only an environment of use for the invention, the MRI device 100 and pacemaker device 200 are not to be understood to be limited to the only devices compatible for use with the present invention implantable lead instrument 10, as the present invention is to be understood to be compatible with any devices and/or components requiring the functionality of the present invention implantable lead instrument 10.

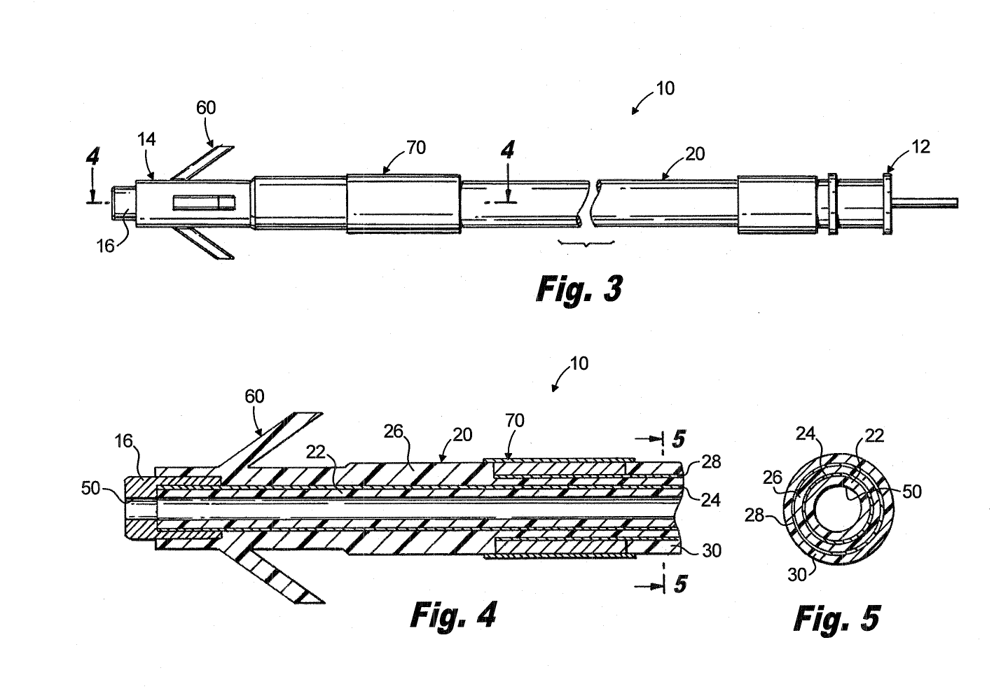

Referring now to FIGS. 3-5, shown is an illustrated embodiment of the present invention medical instrument constructed in accordance with a preferred embodiment of the present invention, designated generally by reference numeral 10. For descriptive purposes, medical instrument 10 is described below as a lead/catheter instrument, however, it is not to be understood to be limited thereto as it relates to any medical device and/or conductor component which is compatible for use with Magnetic Resonance Imaging procedures.

The implantable lead instrument 10 generally consists of a proximal lead connector 12 (adapted and configured to be connected to a separate device, such as a pulse generator 200 (e.g., a pacemaker device 200), and a distal lead tip section 14, preferably adapted and configured for housing one or more electrodes 16 for sensing and causing stimulation of electrical impulses preferably to tissue (e.g., heart tissue) of a patient 110. That is, the lead instrument 10 carries on the proximal side a connector 12 (e.g. IS-1, IS-4, etc.), such that the lead instrument 10 can be connected to a separate device, such as an implantable pulse generator 200 (FIG. 2). Also preferably provided on the distal end section 14 of the lead instrument 10 is a lead fixation mechanism 60 (e.g. Fixation Screw, Fixation Tines etc.) for enabling fixation to body tissue.

As described below, the lead instrument 10 in accordance with the illustrated embodiment of FIGS. 3-5 preferably consists of an elongated tubular member 20, which preferably has several layers of insulation tubing assembled, whereby at least one tubing has on the surface an electrical conductive layer sputtered by a vapor deposition process defined by a non-ferromagnetic conductive material that is compatible with magnetic resonance imaging. The elongate tubular body member 20 is preferably formed of a polymer tubing fabricated from silicon and defines longitudinal axis having opposed proximal and distal end portions. At least one electrical active layer (24, 28) is connected to at least one electrode 16 and the proximal lead connector 12. Provided in a central portion of the elongated tubular member 20 is preferably an open lumen 50 adapted and configured to facilitate passage of a stylet or guidewire type of instrument so as to temporary support the insertion and placement of the lead instrument 10 within a portion of the human body (e.g. heart).

The elongated tubular body member 20 preferably includes a radially inner insulative layer 22, an inner medial conductive layer 24 surrounding the inner insulative layer 22, a medial insulative layer 26 surrounding the inner medial conductive layer 24, an outer medial conductive layer 28 surrounding the medial insulative layer 26 and a radially outer insulative layer 30 surrounding the outer medial conductive layer 28. It is to be understood and appreciated the inner and outer medial conductive layers 24 and 26 are preferably defined by a non-ferromagnetic conductive material that is compatible with magnetic resonance imaging which is applied to at least a portion of the elongated tubular body 20 by a sputtering process. For instance, the sputtered non-ferromagnetic conductive material may be titanium nitrite and iridium oxide, but is not to be understood to be limited thereto.

As mentioned above, further included on device 10 is at least one distal electrode 16 associated with the distal end portion 14 of the elongate tubular body 20 and a proximal electrical connector 12 associated with the proximal end portion of the elongate tubular body 20. Preferably, at least one of the inner 24 and outer 28 conductive layers is electrically connected to the distal electrode 10 and the proximal electrical connector 12. Additionally, the distal lead tip section 14 of elongate tubular body member 20 may include fixation structure for coupling distal electrode 16 to body tissue. Further, the distal electrode 16 and proximal connector 12 are preferably fabricated from a non-ferromagnetic conductive material that is compatible with magnetic resonance imaging, such as titanium or carbon, but not limited thereto.

Further, the lead 10 in accordance with the illustrated embodiment of FIG. 3 is shown to include at least one ring electrode 70 coupled to the outer conductive layer 28 which may be adapted and configured to serve for stimulation of the myocardium in the region of the atrium or for any other suitable purpose. It is also to be appreciated lead 10 may be adapted and configured to include any of a plurality of ring electrodes 70.

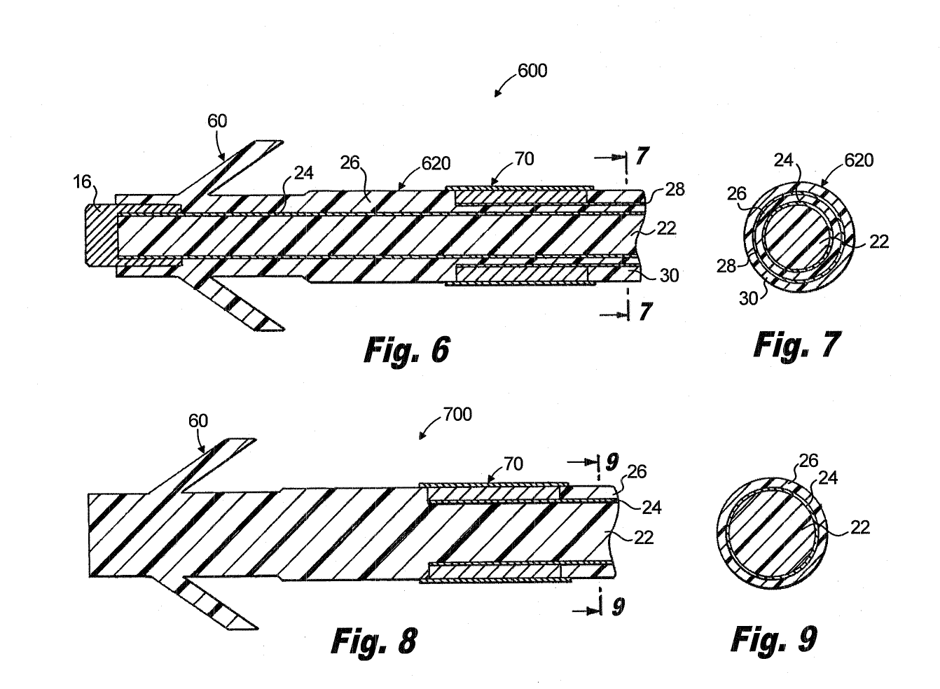

With an illustrated embodiment of the present invention described above in conjunction with FIGS. 3-5, it is to be appreciated and understood this is not to be understood to be the only embodiment of the invention, as the invention encompasses any lead device which is to be used in an MRI, or similar, type of environment. For instance, with reference to FIGS. 6 and 7, shown is a lead device, designated generally by reference numeral 600 which is substantially similar to lead device 10 (FIGS. 3-5) except the elongate tubular body member 620 has a solid core as opposed to being provided with lumen 50. Yet another illustrative embodiment of the invention is depicted on FIGS. 8 and 9 wherein the lead device, designated generally by reference numeral 700, is substantially similar to lead device 600 (FIGS. 6 and 7) except the elongate tubular body member 720 includes the radially inner insulative layer 22, the inner medial conductive layer 24 surrounding the inner insulative layer 22, and the medial insulative layer 26 surrounding the inner medial conductive layer 24. Thus, lead device 700 only includes a single conductive layer (e.g., conductive layer 24), which is shown coupled to the electrode 70 provided about an outer portion of the elongate tubular body member 720.

Therefore, it is to be appreciated advantages of the invention, as described above in accordance with the illustrated embodiment, is the aforesaid sputtered coating is MRI safe and compatible whereby MRI scans will not heat up any of the conductor surfaces of the lead instrument 10. Additionally, the sputtered coating is thinner than a stranded wire or conductor coil and therefore allows the design of smaller leads/catheters mitigating the occurrence of conductor failures caused by breakage of coil or stranded wire, which is a common prior art problem caused with current stimulation leads.

The above presents a description of a best mode contemplated for carrying out the present invention in accordance with the illustrated embodiments and of the process of making the same in such full, clear, concise, and exact terms as to enable any person skilled in the art to which it pertains to make and use these devices and methods. The present invention in accordance with the illustrated embodiments is, however, susceptible to modifications and alternative method steps from those discussed above that are fully equivalent. Consequently, the present invention in accordance with the illustrated embodiments is not limited to the particular embodiments disclosed. On the contrary, the present invention in accordance with the illustrated embodiments are to be understood to cover all modifications and alternative constructions and methods coming within the spirit and scope of the present invention.

What is claimed is:

1. A medical device compatible with magnetic resonance imaging, comprising:
an elongated tubular body defining a longitudinal axis and having opposed proximal and distal end portions, the elongated body including a plurality of coaxial layers including a radially inner-most insulative layer, at least one inner conductive layer, and a radially outer-most insulative layer, wherein the at least one conductive layer is defined by a non-ferromagnetic conductive material that is compatible with magnetic resonance imaging.

2. A medical device as recited in claim 1, wherein the elongate tubular body is formed by a polymer tubing.

3. A medical device as recited in claim 2, wherein the polymer tubing is formed of silicon.

4. A medical device as recited in claim 1, wherein the at least one conductive layer is defined by a conductive material adhered thereto.

5. A medical device as recited in claim 4, wherein the conductive material is applied to the tubular body by a sputtering process.

6. A medical device as recited in claim 4, wherein the conductive material is selected from the group consisting of Titanium Nitrite and Iridium Oxide.

7. A medical device as recited in claim 1, wherein at least one distal electrode is associated with the distal end portion of the tubular body and a proximal electrode is associated with the proximal end portion of the tubular body, and wherein the at least one conductive layer is connected to the distal and proximal electrodes.

8. A medical device as recited in claim 7, wherein the distal and proximal electrodes are constructed from a non-ferromagnetic conductive material selected from the group consisting of Titanium and Carbon.

9. A medical device as recited in claim 1, wherein a central lumen extends through the tubular body for accommodating the passage of a styled or guide wire.

10. A medical device as recited in claim 7, wherein the distal end portion of the tubular body includes a fixation structure for securing the at least distal electrode to tissue.

11. A medical device as recited in claim 7, wherein the proximal electrode is defined by a connector selected from the group consisting of an IS-1 connector, IS-4 connector and LV-1 connector.

12. A medical device as recited in claim 7, wherein the distal and proximal electrodes are constructed from a non-ferromagnetic conductive material selected from the group consisting of Titanium and Carbon.

13. A medical device compatible with magnetic resonance imaging, comprising:
an elongated tubular body defining a longitudinal axis and having opposed proximal and distal end portions, the elongated body including:
a) a radially inner insulative layer;
b) an inner medial conductive layer surrounding the inner insulative layer;
c) a medial insulative layer surrounding the inner medial conductive layer;
d) an outer medial conductive layer surrounding the medial insulative layer wherein the inner and outer medial conductive layers are defined by a non-ferromagnetic conductive material that is compatible with magnetic resonance imaging and is applied to a portion of the elongated tubular body by a sputtering process;
e) a radially outer insulative layer surrounding the outer medial conductive layer; and
f) at least one distal electrode is associated with the distal end portion of the tubular body and a proximal electrode is associated with the proximal end portion of the tubular body, and wherein at least one conductive layer is connected to the distal and proximal electrodes.

14. A medical device as recited in claim 13, wherein the conductive material of each inner and outer medial conductive layer is selected from the group consisting of Titanium Nitrite and iridium Oxide.

15. A medical device as recited in claim 13, wherein the elongate tubular body is formed by a polymer tubing.

16. A medical device as recited in claim 15, wherein the polymer tubing is formed of silicon.

17. A medical device as recited in claim 15, wherein the distal and proximal electrodes are constructed from a non-ferromagnetic conductive material selected from the group consisting of Titanium and Carbon.

18. A medical device as recited in claim 13, wherein a central lumen extends through the tubular body for accommodating the passage of a styled or guide wire.

19. A medical device as recited in claim 13, wherein the distal end portion of the tubular body includes a fixation structure for securing the at least distal electrode to tissue.

20. A method of producing a medical device compatible with magnetic resonance imaging comprising the steps of:
providing elongated tubular body defining a longitudinal axis and having opposed proximal and distal end portions, the elongated body including a plurality of coaxial layers;
sputtering at least coaxial layer with a non-ferromagnetic conductive material that is compatible with magnetic resonance imaging to provide at least one conductive layer in the elongated tubular body;
providing at least one electrode on at least one of the proximal and distal ends portions of the elongated tubular body constructed from a non-ferromagnetic conductive material; and
electrically connecting the at least one electrode with the at least one conductive layer.
